(19) United States
(12) Patent Application Publication
(10) Pub. No.: US 2021/0285947 A1
(43) Pub. Date: Sep. 16, 2021

(54) MULTIANALYTE ASSAY CARTRIDGES AND SYSTEM THAT USES THE SAME

(71) Applicant: Khanh Bui, San leandro, CA (US)

(72) Inventor: Khanh Bui, San leandro, CA (US)

(21) Appl. No.: 17/199,214

(22) Filed: Mar. 11, 2021

Related U.S. Application Data
(60) Provisional application No. 62/987,966, filed on Mar. 11, 2020.

(51) Int. Cl. G01N 33/543

(52) U.S. Cl. CPC G01N 33/54386

(57) ABSTRACT

An assay cartridge for sensing multiple analytes includes a capture platform and main body. The capture platform includes appendages. The main body includes first, second, and third assemblies of reaction compartments as well as first and second wash basins. The main body includes a capture platform cradle that supports the capture platform. Each appendage is coupled to a capture agent that specifically binds to an analyte. The first reaction compartments each comprise a matrix that includes the analyte. The first wash basin includes a first wash solution that removes non-specific binding between the analyte and capture agent. The second reaction compartments each include a detection antibody. The second wash basin includes a second wash solution that remove non-specific binding of the detection antibody to the analyte. The third reaction compartments each include a substrate that reacts with a conjugated enzyme of the detection antibody and changes color as a result.

### CROSS-REFERENCE TO RELATED APPLICATIONS

This application claims priority to U.S. Provisional Application No. 62/987,966 filed Mar. 11, 2020, which is hereby incorporated herein by reference.

### FIELD OF THE INVENTION

The present disclosure relates generally to assay cartridges. More specifically, the present disclosure describes multianalyte assay cartridges.

### BACKGROUND OF THE INVENTION

Current Point of Care Testing (“POCT”) typically use quick, fast lateral flow immuno-precipitation method to qualitatively identify presence of individual biomarkers with little quantitative value. For example, in centralized laboratories, high quality testing is performed on automated instruments that are expensive to own by smaller structures and require extensive training and highly experienced specialists to operate. Multiplexing systems have been developed in an attempt to increase efficiency and reduce cost for the system. However, they are still too expensive and do not answer the accessibility issue due to requirements of supporting infrastructures and extensive training on highly experienced specialist. A multiplex system, for example, might cost from $27,500 to over $100,000. In addition, extended turn-around times and mismanagement of laboratory testing have also been issues for patients who would have to come back to the doctors for results.

### BRIEF DESCRIPTION OF THE DRAWINGS

Some of the embodiments will be described in detail, with reference to the following figures, wherein like designations denote like members, wherein:

FIG. 1 depicts a perspective view of an assay cartridge, according to some embodiments.

FIG. 2 depicts a top view of the main body of the assay cartridge, according to other embodiments.

FIG. 3 illustrates View A of FIG. 1, according to certain embodiments.

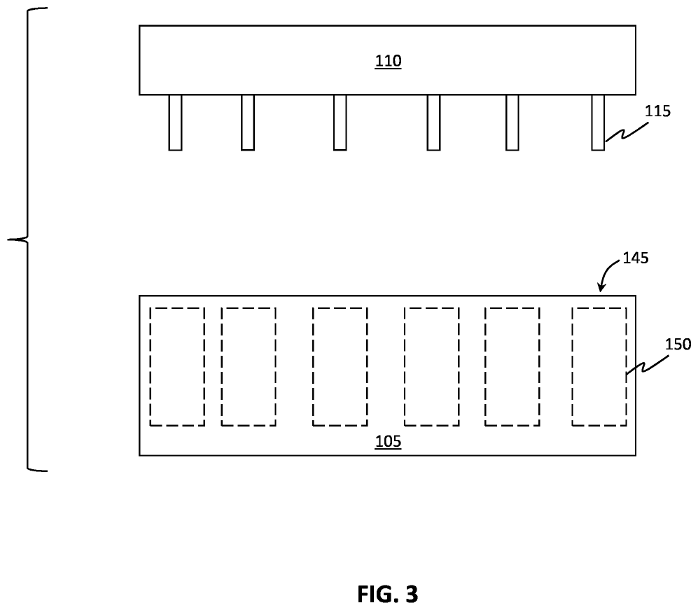

FIG. 4 illustrates a system, according to yet still other embodiments.

FIG. 5 illustrates the various positions that a capture platform can assume relative to the assay cartridge, according to some embodiments.

FIG. 6 illustrates various reaction products, according to other embodiments.

FIG. 7 illustrates the process steps of a method, according to certain embodiments.

FIG. 8 illustrates a block diagram of components of a data processing system, according to yet still other embodiments.

Unless otherwise specifically noted, articles depicted in the drawings are not necessarily drawn to scale.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate

The present disclosure includes many aspects and features. Moreover, while many aspects and features relate to, and are described in the context of assay cartridges, embodiments of the present disclosure are not limited to use only in this context.

Current Point of Care Testing (“POCT”) typically use quick, fast lateral flow immuno-precipitation method to qualitatively identify presence of individual biomarkers with little quantitative value. For example, in centralized laboratories, high quality testing is performed on automated instruments that are expensive to own by smaller structures and require extensive training and highly experienced specialists to operate. Multiplexing systems have been developed in an attempt to increase efficiency and reduce cost for the system. However, they are still too expensive and do not answer the accessibility issue due to requirements of supporting infrastructures and extensive training on highly experienced specialist. A multiplex system, for example, might cost from $27,500 to over $100,000. In addition, extended turn-around times and mismanagement of laboratory testing have also been issues for patients who would have to come back to the doctors for results.

There exists a need for a POCT technology that is portable, versatile, and capable of providing quantitative analysis of biomarkers with accuracy, sensitivity, and specificity. The POCT technology should emphasize cost reduction, accuracy, sensitivity, and specificity. The traditional multiplexed immunoassay utilizes antibodies that are “fixed” to a support (e.g., microtiter plate well surface) and reagents that are delivered into and out of the same well of the microtiter plate for each same. Here, all reactions occur within the same well, which means assay parameters are harmonized across all assays, which is a potential for crosstalk or non-specific binding that could cause random signals.

The instant disclosure seeks to provide an assay cartridge that utilizes reagents that are “fixed” within pre-filled compartments and requires that the capture antibodies conjugated onto the surface of a mobile capture platforms moved into and out of reagent compartments. The instant disclosure seeks to provide a process to run multianalyte assays that are either fully automated or manual depending on applications. The instant disclosure seeks to provide a system that only requires that the user load the assay cartridge and sample (e.g., for point-of-care applications). The instant disclosure seeks to provide an assay cartridge where each assay occurs in a dedicated reaction compartment such that reaction parameters can be optimized for each assay (i.e., no crosstalk). The instant disclosure further seeks to provide assay cartridges for sensing multiple analytes in a sample by performing multiple simultaneous real-time assays on the sample.

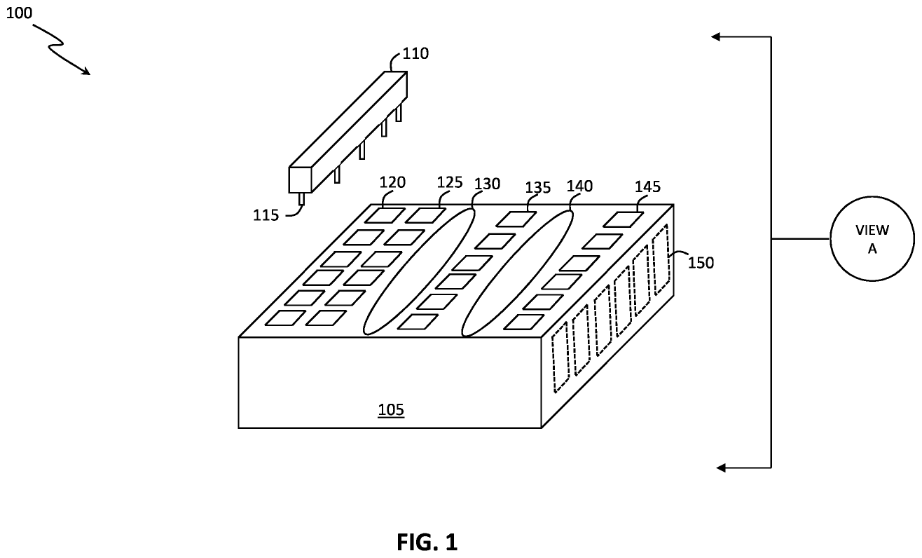

The FIGS. are included herein to facilitate the discussion of the instant disclosure. FIG. 1 depicts a perspective view of an assay cartridge for sensing multiple analytes, generally 100, in accordance with some embodiments. The assay cartridge is preferably made of plastic or similar polymers. The assay cartridge 100 includes a capture platform 110 and a main body 105. The capture platform 110 includes a plurality of appendages 115. The main body 105 preferably has a flat polygonal body structure and includes a first assembly of reaction compartments 125, a second assembly of reaction compartments 135, a third assembly of reaction compartments 145, a first wash basin 130, a second wash basin 140, and a capture platform cradle 120 that is adapted to support the capture platform 110 as well as individually house each appendage 115. For example, the capture platform cradle 120 includes compartments that each house one of the plurality of appendage 115.

Similar to microtiter plates known in the art, the reaction compartments of the main body 105 are substantially arranged in a grid of rows and columns that are interrupted by the first wash basin 130 and the second wash basin 140. The columns are represented by the first assembly of reaction compartments 125, the second assembly of reaction compartments 135, and the third assembly of reaction compartments 145. According to preferred embodiments, each column represents an assay stage and each row represents an individual assay that progresses the first assembly of reaction compartments 125 to the third assembly of reaction compartments 145. The main body 105 also includes voids 150 that are positioned on a side thereon and proximate to the third assembly of reaction compartments 145. The voids 150 facilitate reading (i.e., detecting the intensity of transmitted light using the detector 420 which is a spectrophotometer) sample reactions in each of the compartments of the third assembly of reaction compartments 145. Each appendage 115 preferably includes a capture agent (e.g., capture agent 605) bound thereto that is adapted to specifically bind to an analyte (e.g., analyte 610). Although capture agents 605 are preferably antibodies, other types can be employed.

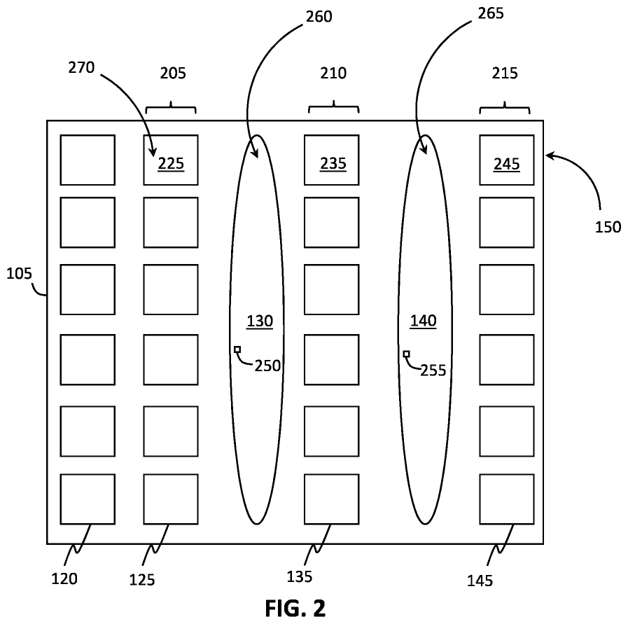

FIG. 2 depicts a top view of the main body 105, according to some embodiments. The first assembly of reaction compartments 125 are preferably serially arranged in a first column 205 and each include a sample, which consists of a matrix 270 that includes the analyte(s) (e.g., analyte 610). Applicable matrices include, but are not limited to, serum, plasma, whole blood, urine, cell lysates, and any other media, secretions, or samples that contain biomarkers. For example, a first reaction compartment 225 of the first assembly of reaction compartments 205 is adapted to receive an appendage 115 (of the plurality of appendages) and thereby facilitate specific binding of the analyte 610 to the capture agent 605. The first wash basin 130 is adapted to receive the plurality of appendages 115 and includes a first wash solution 260 (e.g., a wash buffer) that removes non-specific binding of the analyte 610 to the capture agent 605.

The first wash basin 130 is preferably located proximate to the first assembly of reaction compartments 205 and the second assembly of reaction compartments 210. The first wash basin 130 includes a first agitating component 250 that agitates the first wash solution 260 when actuated and thereby produce a vigorous washing event. Although many different types of components can be used to agitate the first wash solution, the first agitating component 250 is preferably a magnetic stir bar that is rotated (i.e., actuated) via a rotating magnetic field (e.g., produced by an agitator 415). The second assembly of reaction compartments 135 are preferably serially arranged in a second column 210 and each compartment includes a detection antibody 615. The detection antibody 615 is conjugated with an enzyme. For example, the enzyme can be horseradish peroxidase (“HRP”). HRP is an enzyme used to amplify signal photometric assays by catalyzing the conversion of chromogenic or chemiluminescent substrates for the detection of targets (e.g., proteins, carbohydrates, etc.). In general, the enzyme can be any enzyme that can catalyze the conversion of chromogenic or chemiluminescent substrates for the detection of targets, in accordance with certain embodiments. A second reaction compartment 235 of the second assembly of reaction compartments 135 is adapted to receive the appendage 115 and thereby facilitate specific binding of the detection antibody 615 to the analyte 610.

The second wash basin 140 is similar structurally and functionally to the first wash basin 130 and is adapted to receive the plurality of appendages 115 and includes a second wash solution 265 that remove non-specific binding of the detection antibody 615 to the analyte 610. The second wash basin 140 is preferably located proximate to the second assembly of reaction compartments 135 and the third assembly of reaction compartments 145. The second wash basin 140 includes a second agitating component 225 that agitates the second wash solution 265 when actuated. The second agitating component 225 is structurally and functionally similar to the first agitating component 250.

The third assembly of reaction compartments 145 are preferably serially arranged in a third column 215 each compartment includes a substrate (e.g., substrate 620). Different types of substrates may be used in conjunction with different types of enzymes that may be conjugated to the detection antibody 615 (e.g., 3,3′, 5,5-tetramethylbenzidine or TMB or equivalent may be used with HRP). Applicable substrates that can be used with HRP include, but are not limited to, 2,2′-Azino-bis(3-ethylbenzthiazoline-6-sulfonic acid) (“ABTS”), o-phenylenediamine dihydrochloride (“OPD”), and similar substrates. In general, substrate 620 can be any chromogenic substrate that can react with the enzyme conjugated to the detector antibody 615. Although other arrangements and orientations are possible, the first column 205, the second column 210, and the third column 215 are preferably oriented parallel to each other.

Each of the first column 205, the second column 210, and the third column 215 include reaction compartments that are serially aligned such that each reaction compartment of a column has counterparts in the other columns. The substrate 620 is adapted to change color when it reacts with the enzyme conjugated to the detection antibody 615. For example, the substrate 620 is enzymatically cleaved by HRP to change color from clear to blue when the HRP-conjugated detection antibody (detection antibody 615) is submerged into the substrate solution (e.g., of the third assembly of reaction compartments 145). The third assembly of reaction compartments 145 includes a plurality of third reaction compartments 245. A third reaction compartment 245 of the third assembly of reaction compartments 145 is adapted to receive one of the appendages 115 and thereby facilitate and thereby facilitate a reaction of the substrate 620 with the conjugated enzyme 625 (e.g., HRP) of the detection antibody 615. In other words, each reaction compartment of the third assembly of reaction compartments 145 is adapted to receive an appendage 115. The conjugated enzyme 625 (e.g., HRP) reacts with the substrate 620 (e.g., TMB) and thereby changes the color of the substrate 620 from a first color state (e.g., substrate 620a) to a second color state (e.g., substrate 620b). For example, HRP reacts with TMB and thereby turns the color of the substrate from clear to blue. The reaction of the conjugated enzyme 625 with the substrate 620 can be stopped by removing the appendages 115 from the third assembly reaction compartments 145. The substrate color changes in each of the third assembly reaction compartments 145 are subsequently read by a detector 420 (discussed below).

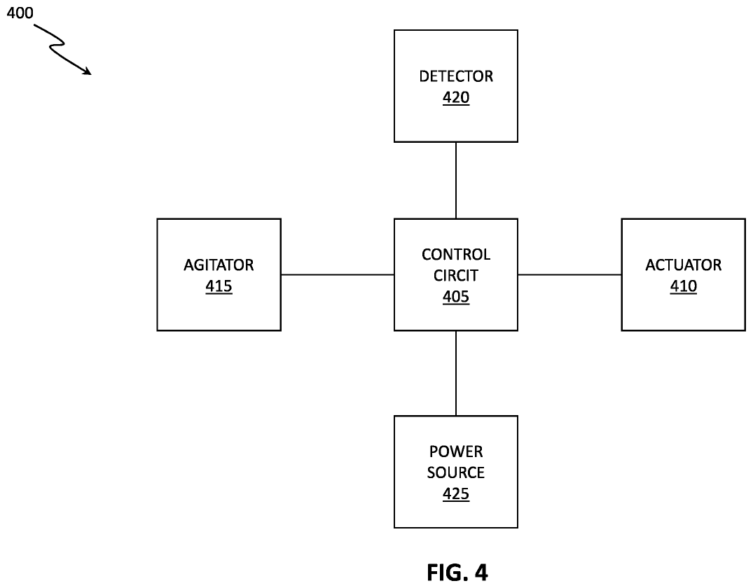

FIG. 4 depicts a system, generally 400, for sensing multiple analytes, according to certain embodiments. The system 400 preferably includes an actuator 410, an agitator 415, a detector 420, and a power source 425 all communicatively coupled to each other via the control circuit 405. To be sure, the control circuit 405 can be one or more devices that are configured to perform one or more process, steps, functions that are included in the instant disclosure. The actuator 410 is a mechanical assembly that is adapted to move the capture platform 110 about the main body 105 to perform the assay disclosed herein. In other words, the capture platform 110 is a dynamically positioned component that traverses the statically positioned main body 105 via the actuator 410. Alternatively, the main body 105 can be the dynamically positioned component and the capture platform 110 can be statically positioned.

The agitator 415 is a device that actuates the first agitating component 250 and the second agitating component 255 and thereby agitates the first wash solution 260 and the second wash solution 265. The detector 420 is a spectrophotometer or device capable of quantitative measurement of the reflection or transmission properties of a material as a function of wavelength. The power source 425 can be any source of electrical power sufficient to power the system 100.

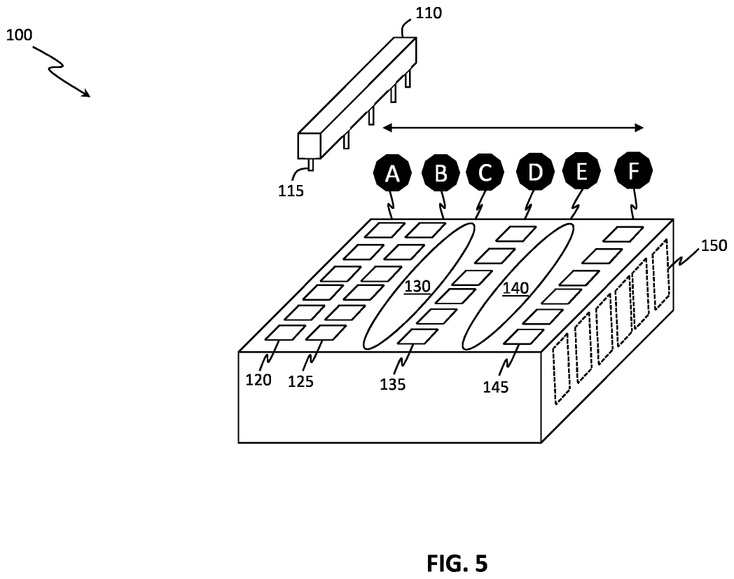

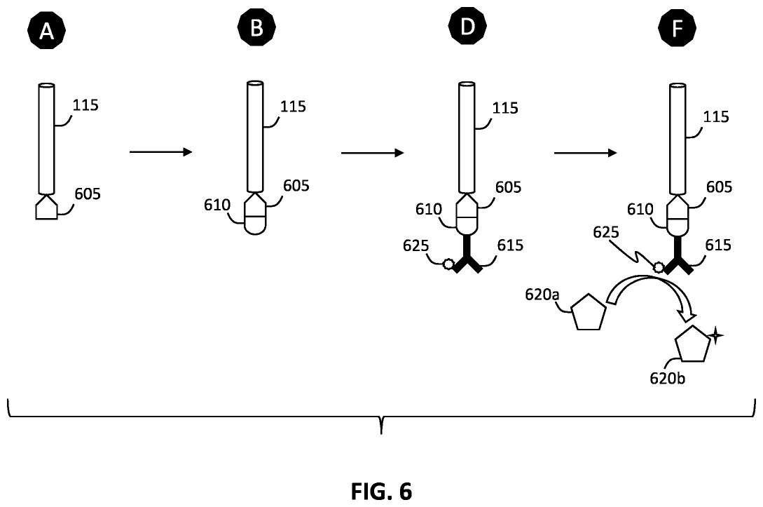

FIG. 5 depicts the assay cartridge 100, according to other embodiments. To perform the assay, the capture platform 110 assumes Positions A-F. To perform a multianalyte assay, the capture platform 110 preferably assumes the aforementioned positions in the following order: Position A; Position B; Position C; Position D; Position C; Position E; and Position F. Position A aligns with the capture platform cradle 120. Position B aligns with the first assembly of reaction compartments 125 (i.e., the analytes). Position C aligns with the first wash basin 130 (i.e., the wash buffer). Position D aligns with the second assembly of reaction compartments 135 (i.e., the detector antibody). Position E aligns with the second wash basin 140 (i.e., the wash buffer). Position F aligns with the third assembly of reaction compartments 145 (i.e., the substrate). FIG. 6 depicts the various structures and reaction products that are present at Positions A, B, D, and F.

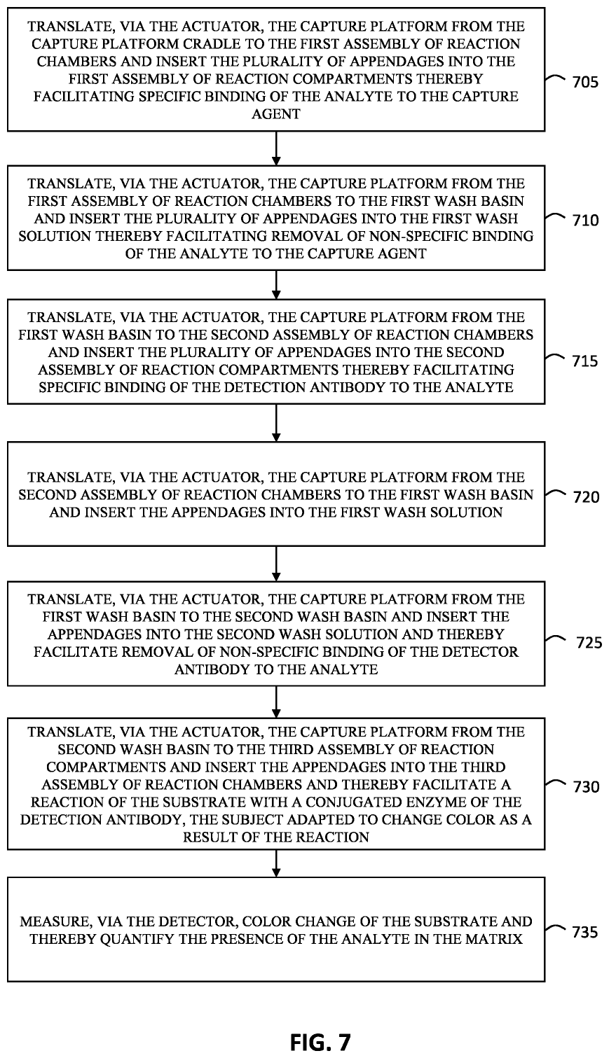

FIG. 7 depicts a method, according to yet still other embodiments. Samples are added to each compartment of the first assembly of reaction chambers 125. Samples preferably consist of one or more analytes 610 suspended in a matrix 270 solution. The plurality of appendages 115 each have one or more capture agent 605. At Step 705, the actuator 410 translates the capture platform 110 from the capture platform cradle 120 to the first assembly of reaction chambers 125 and inserts the plurality of appendages 115 into the first assembly of reaction compartments 125 thereby facilitating specific binding of the analyte 610 to the capture agent 605. Here, the capture platform 110 moves from Position A to Position B. At Step 710, the actuator 410 translates the capture platform 110 from the first assembly of reaction chambers 125 to the first wash basin 130 and inserts the plurality of appendages 115 into the first wash solution 260 thereby facilitating removal of non-specific binding of the analyte 610 to the capture agent 605. Here, the capture platform 110 moves from Position B to Position C. For example, the first wash solution 260 is preferably circulated, stirred, swirled, agitated, etc. via the first agitating component 250, which is activated via the agitator 415.

At position 715, the actuator 410 translates the capture platform 110 from the first wash basin 260 to the second assembly of reaction chambers 135 and inserts the plurality of appendages 115 into the second assembly of reaction compartments 135 and thereby facilitates specific binding of the detection antibody 615 to the analyte 610 (see the reaction product of Position D). Here, the capture platform 110 moves from Position C to Position D. At Step 720, the actuator 410 translates the capture platform 110 from the second assembly of reaction chambers 135 to the first wash basin 130 and inserts the appendages 115 into the first wash solution 260. Here, the capture platform 130 moves from Position D to Position C. At Step 725, the actuator 410 translates the capture platform 110 from the first wash basin 130 to the second wash basin 140 and inserts the appendages 115 into the second wash solution 265 and thereby facilitate removal of non-specific binding of the detector antibody 615 to the analyte 610. Here, the capture platform 110 moves from Position C to Position E.

At Step 730, the actuator 410 translates the capture platform 110 from the second wash basin 140 to the third assembly of reaction compartments 215 and inserts the appendages 115 into the third assembly of reaction chambers 215 and thereby facilitate a reaction of the substrate 620 with a conjugated enzyme 625 of the detection antibody 615. As discussed above, the substrate 620 is adapted to change color as a result of the reaction. Here, the capture platform 110 moves from Position E to Position F and yields a reaction product that results from the reaction of the substrate 620 with the conjugated enzyme 625, as depicted in FIG. 6. At Step 735, the detector 420 measures the color change of the substrate 620 and thereby quantifies the presence of the analyte 610 in the matrix 270.

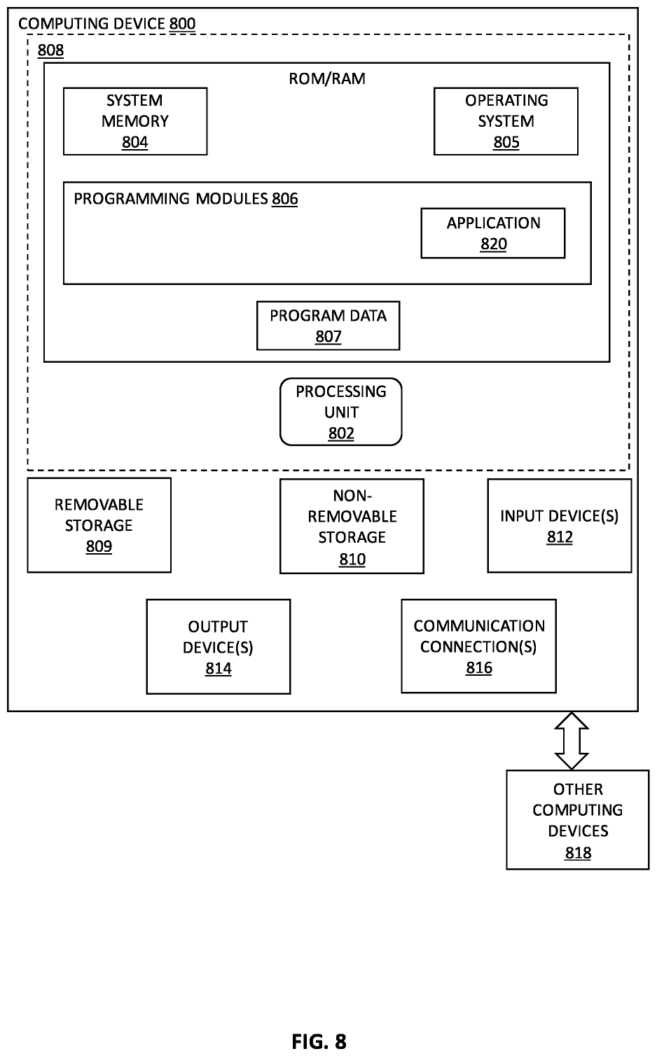

With reference to FIG. 8, a system consistent with an embodiment of the disclosure may include a computing device or cloud service, such as computing device 800. Computing device 800 can represent system 400. In a basic configuration, computing device 800 may include at least one processing unit 402 (e.g., control circuit 112) and a system memory 804. Depending on the configuration and type of computing device, system memory 804 may comprise, but is not limited to, volatile (e.g., random-access memory (RAM)), non-volatile (e.g., read-only memory (ROM)), flash memory, or any combination. System memory 804 may include operating system 805, one or more programming modules 806, and may include a program data 807. Operating system 805, for example, may be suitable for controlling computing device 800's operation. In one embodiment, programming modules 806 may include machine learning module. Furthermore, embodiments of the disclosure may be practiced in conjunction with a graphics library, other operating systems, or any other application program and is not limited to any particular application or system. This basic configuration is illustrated in FIG. 8 by those components within a dashed line 808.

Computing device 800 may have additional features or functionality. For example, computing device 800 may also include additional data storage devices (removable and/or non-removable) such as, for example, magnetic disks, optical disks, or tape. Such additional storage is illustrated in FIG. 8 by a removable storage 809 and a non-removable storage 810. Computer storage media may include volatile and nonvolatile, removable, and non-removable media implemented in any method or technology for storage of information, such as computer-readable instructions, data structures, program modules, or other data. System memory 804, removable storage 809, and non-removable storage 810 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 800.

Computing device 400 may also have input device(s) 812 such as a keyboard, a mouse, a pen, a sound input device, a touch input device, a location sensor, a camera, a biometric sensor, etc. Output device(s) 814 such as a display, speakers, a printer, etc. may also be included. The aforementioned devices are examples and others may be used.

Computing device 800 may also contain a communication connection 816 that may allow device 800 to communicate with other computing devices 818, such as over a network in a distributed computing environment, for example, an intranet or the Internet. Communication connection 816 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 804, including operating system 805. While executing on processing unit 802 (e.g., control circuit 405), programming modules 806 (e.g., application 820 such as a media player) may perform processes including, for example, one or more stages of methods, algorithms, systems, applications, servers, databases as described above. The aforementioned process is an example, and processing unit 802 (e.g., control circuit 405) may perform other processes. Other programming modules that may be used in accordance with embodiments of the present disclosure may include machine learning application.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the disclosure has been explained in relation to its preferred embodiment, it is to be understood that many other possible modifications and variations can be made without departing from the spirit and scope of the disclosure.

1. An assay cartridge for sensing multiple analytes, the cartridge comprising:
a capture platform comprising a plurality of appendages;
a main body comprising:
a first assembly of reaction compartments;
a second assembly of reaction compartments;
a third assembly of reaction compartments;
a first wash basin;
a second wash basin;
a capture platform cradle adapted to support the capture platform;
wherein
each appendage comprises a capture agent bound thereto, the capture agent adapted to specifically bind to an analyte;
the first assembly of reaction compartments each comprise a matrix, the matrix comprises the analyte, a first reaction compartment of the first assembly of reaction compartments is adapted to receive an appendage and thereby facilitate specific binding of the analyte to the capture agent;
the first wash basin is adapted to receive the plurality of appendages and comprises a first wash solution that removes non-specific binding of the analyte to the capture agent;
the second assembly of reaction compartments each comprise a detection antibody, a second reaction compartment of the second assembly of reaction compartments is adapted to receive the appendage and thereby facilitate specific binding of the detection antibody to the analyte;
the second wash basin is adapted to receive the plurality of appendages and comprises a second wash solution that remove non-specific binding of the detection antibody to the analyte; and
the third assembly of reaction compartments each comprise a substrate, a third reaction compartment of the third assembly of reaction compartments is adapted to receive the appendage and thereby facilitate a reaction of the substrate with a conjugated enzyme of the detection antibody, the substrate is adapted to change color as a result of the reaction.

2. The assay cartridge of claim 1, wherein
the first wash basin is located proximate to the first assembly of reaction compartments and the second assembly of reaction compartments.

3. The assay cartridge of claim 2, wherein
the second wash basin is located proximate to the second assembly of reaction compartments and the third assembly of reaction compartments.

4. The assay cartridge of claim 3, wherein
the capture platform is adapted to be actuated by an actuator.

5. The assay cartridge of claim 4, wherein
the first wash basin comprises a first agitating component that agitates the first wash solution when actuated.

6. The assay cartridge of claim 5, wherein
the second wash basin comprises a second agitating component that agitates the second wash solution when actuated.

7. The assay cartridge of claim 6, wherein
the first assembly of reaction compartments are serially arranged in a first column;
the second assembly of reaction compartments are serially arranged in a second column;
the third assembly of reaction compartments are serially arranged in a third column; and
the first column, the second column, and the third column are oriented parallel to each other.

8. The assay cartridge of claim 6 included in a system, the system comprising:
an actuator;
an agitator;
a detector;
a power source;
a control circuit communicatively coupled to the actuator, the agitator, the detector, and the power source; and
wherein the control circuit is configured to quantify a presence of the analyte in the matrix.

9. The assay cartridge of claim 8, wherein in quantifying the analyte, the control circuit is configured to:
translate, via the actuator, the capture platform from the capture platform cradle to the first assembly of reaction chambers and insert the plurality of appendages into the first assembly of reaction compartments thereby facilitating specific binding of the analyte to the capture agent.

10. The assay cartridge of claim 9, wherein in quantifying the analyte, the control circuit is configured to:
translate, via the actuator, the capture platform from the first assembly of reaction chambers to the first wash basin and insert the plurality of appendages into the first wash solution thereby facilitating removal of non-specific binding of the analyte to the capture agent.

11. The assay cartridge of claim 10, wherein in quantifying the analyte, the control circuit is configured to:
translate, via the actuator, the capture platform from the first wash basin to the second assembly of reaction chambers and insert the plurality of appendages into the second assembly of reaction compartments thereby facilitating specific binding of the detection antibody to the analyte.

12. The assay cartridge of claim 11, wherein in quantifying the analyte, the control circuit is configured to:
translate, via the actuator, the capture platform from the second assembly of reaction chambers to the first wash basin and insert the appendages into the first wash solution.

13. The assay cartridge of claim 12, wherein in quantifying the analyte, the control circuit is configured to:
translate, via the actuator, the capture platform from the first wash basin to the second wash basin and insert the appendages into the second wash solution and thereby facilitate removal of non-specific binding of the detector antibody to the analyte.

14. The assay cartridge of claim 13, wherein
in quantifying the analyte, the control circuit is configured to:
translate, via the actuator, the capture platform from the second wash basin to the third assembly of reaction compartments and insert the appendages into the third assembly of reaction chambers and thereby facilitate a reaction of the substrate with a conjugated enzyme of the detection antibody;
the substrate is adapted to change color as a result of the reaction; and
measure, via the detector, color change of the substrate and thereby quantify the presence of the analyte in the matrix.

14. (canceled)

15. The assay cartridge of claim 14, wherein
the agitator actuates one or more of
the first agitating component and thereby agitates the first wash solution; and
the second agitating component and thereby agitates the second wash solution.

16. An assay cartridge for sensing multiple analytes, the cartridge comprising:
a capture platform comprising a plurality of appendages;
a main body comprising:
a first assembly of reaction compartments;
a second assembly of reaction compartments;
a third assembly of reaction compartments;
a first wash basin;
a second wash basin;
wherein
each appendage comprises a capture agent bound thereto, the capture agent adapted to specifically bind to an analyte;
the first assembly of reaction compartments each comprise a matrix, the matrix comprises the analyte, a first reaction compartment of the first assembly of reaction compartments is adapted to receive an appendage and thereby facilitate specific binding of the analyte to the capture agent;
the first wash basin is adapted to receive the plurality of appendages and comprises a first wash solution that removes non-specific binding of the analyte to the capture agent;
the second assembly of reaction compartments each comprise a detection antibody, a second reaction compartment of the second assembly of reaction compartments is adapted to receive the appendage and thereby facilitate specific binding of the detection antibody to the analyte;
the second wash basin is adapted to receive the plurality of appendages and comprises a second wash solution that remove nonspecific binding of the detection antibody to the analyte; and
the third assembly of reaction compartments each comprise a substrate, a third reaction compartment of the third assembly of reaction compartments is adapted to receive the appendage and thereby facilitate a reaction of the substrate with a conjugated enzyme of the detection antibody, the substrate is adapted to change color as a result of the reaction.

17. The assay cartridge of claim 16, wherein
the capture platform is adapted to be actuated by an actuator.

18. The assay cartridge of claim 16, wherein
the first wash basin comprises a first agitating component that agitates the first wash solution when actuated.

19. The assay cartridge of claim 16, wherein
the second wash basin comprises a second agitating component that agitates the second wash solution when actuated.

20. The assay cartridge of claim 16, wherein
the first assembly of reaction compartments are arranged in a first column;
the second assembly of reaction compartments are arranged in a second column;
the third assembly of reaction compartments are arranged in a third column; and
the first column, the second column, and the third column are oriented parallel to each other.
